(19) United States
(12) Plant Patent Application Publication
(10) Pub. No.: US 2004/0250326 P1
(43) Pub. Date: Dec. 9, 2004

(54) New Guinea impatiens plant named 'Fisnics Zorred'

(75) Inventor: Birgit C. Hofmann, Bendorf (DE)

Correspondence Address: FOLEY AND LARDNER, SUITE 500, 3000 K STREET NW, WASHINGTON, DC, 20007, US

(73) Assignee: Florfis AG

(21) Appl. No.: 10/829,347

(22) Filed: Apr. 22, 2004

(30) Foreign Application Priority Data
Apr. 22, 2003 (CH) 03-1993

(51) Int. Cl. A01H 5/00

(52) U.S. Cl. PLT/318

(57) ABSTRACT

A new and distinct cultivar of New Guinea Impatiens plant named ‘Fisnics Zorred’, particularly characterized by the combined features of brilliant, orange-red flower color; large, round flowers, flat to slightly cup-shaped; intense medium green foliage, and long, narrow leaves; medium growth habit, round, dense and very well branched plant habit; early flowering response, and floriferous throughout the summer; and suitable both for early sale as flowering pot plant and for outdoor bedding plant.

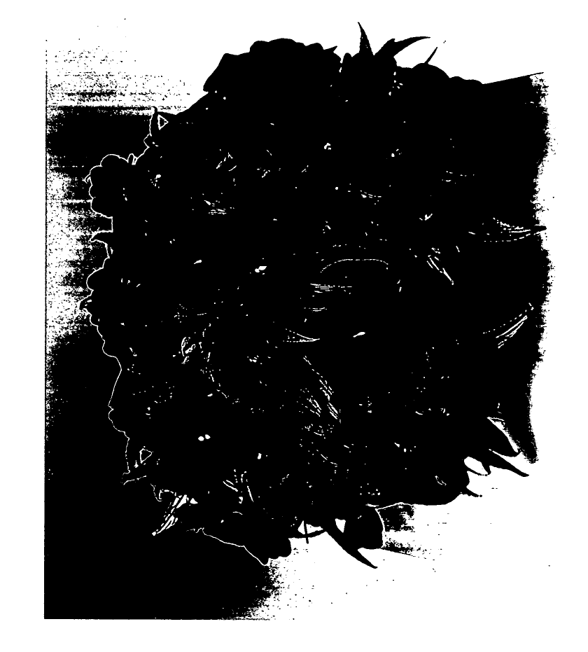

### GENUS AND SPECIES OF THE PLANT CLAIMED

Impatiens hawkeri W. Bull (hybrid)

### VARIETY DENOMINATION

Fisnics Zorred

### BACKGROUND OF THE INVENTION

The present invention comprises a new and distinct cultivar of New Guinea Impatiens, botanically known as Impatiens hawkeri W.Bull, and hereinafter referred to by the cultivar name ‘Fisnics Zorred’.

‘Fisnics Zorred’ is a product of a planned breeding program and originated from a hybridization made by the inventor, Birgit C. Hofmann, in a controlled breeding program in Hillscheid, Germany, in 2000.

The female parent was the variety ‘Harmony Super Red’ (unpatented), characterized by deep red, round flowers, medium green foliage, and about medium sized, relatively dense plant habit.

The male parent was the variety ‘Fisnics Scarlet Blush’ (U.S. Plant Pat. No. 13,227), having scarlet flowers with a small purple eye at the center, dark green leaves with reddish under side, and compact to medium growth habit.

‘Fisnics Zorred’ was discovered and selected as one flowering plant within the progeny of the stated cross by the inventor in April 2001 in a greenhouse in Galdar, Canary Islands, Spain.

The first act of vegetative or asexual reproduction of ‘Fisnics Zorred’ was accomplished when cuttings were taken from the initial selection in July 2001 in a controlled environment in Galdar, Gran Canaria, Spain, by, or under the supervision of the inventor.

Horticultural examination of plants grown from these cuttings initiated in spring of 2002 in Hillscheid, Germany, and continuing thereafter, has demonstrated that the combination of characteristics as herein disclosed for ‘Fisnics Zorred’ are firmly fixed and are retained through successive generations of asexual reproduction.

‘Fisnics Zorred’ has not been observed under all possible environmental conditions. The phenotype may vary significantly with variations in environment such as temperature, light intensity and day length, without, however, any variation in genotype. The following observations, measurements, and comparisons describe plants grown in Hillscheid, Federal Republic of Germany, under greenhouse conditions which approximate those generally used in commercial practice.

### BRIEF SUMMARY OF THE INVENTION

The following traits have been repeatedly observed and are determined to be basic characteristics of ‘Fisnics Zorred’, which in combination distinguish this Impatiens as a new and distinct cultivar:

1. brilliant, orange-red flower color;

2. large, round flowers, flat to slightly cup-shaped;

3. intense medium green foliage, and long, narrow leaves;

4. medium growth habit, round, dense and very well branched plant habit;

5. early flowering response, and floriferous throughout the summer; and

6. suitable both for early sale as flowering pot plant and for outdoor bedding plant.

Of the many commercial cultivars known to the inventor, the most similar in comparison to ‘Fisnics Zorred’ is the parental variety ‘Harmony Super Red’, and the commercial variety ‘Celebrette Scarlet’ (U.S. Plant Pat. No. 10,110).

In comparison to ‘Harmony Super Red’, ‘Fisnics Zorred’ has flowers that are flatter in shape and have a somewhat less pure red hue, and more orange-red in color. Furthermore, the leaves of ‘Fisnics Zorred’ are unusually narrow in shape, while ‘Harmony Super Red’ has distinctly wider, elliptically to ovate shaped leaves.

In comparison to ‘Celebrette Scarlet’, ‘Fisnics Zorred’ has a similar flower color, but larger flowers, longer and narrower leaves, and less compact plant habit.

### BRIEF DESCRIPTION OF THE DRAWINGS

The accompanying photographic drawing shows typical flower and foliage characteristics of ‘Fisnics Zorred’ with colors being as true as possible with illustrations of this type. The photographic drawing shows a side view of a typical flowering plant of ‘Fisnics Zorred’.

### DETAILED BOTANICAL DESCRIPTION

In the following description color references are made to The Royal Horticultural Society Colour Chart (RHS). The color values were determined indoors from plants grown in Hillscheid, Federal Republic of Germany, under greenhouse conditions which approximate those generally used in commercial practice.

The description is based on plants which were planted as rooted cuttings in 12 cm pots in late February 2003, and then grown in the greenhouse at 16° C. minimum temperature. Most observations and measurements were made after the beginning of flowering in mid May, when the plants were about 12 weeks old.

### PLANT

General appearance and form:

Plant habit.—Medium sized, rounded foliage canopy, dense and very well-branched; growth is indeterminate, though weak after begin of flowering.

Height.—17.9 cm.

Width.—29.0 cm.

Number of branches.—14.5.

Length of branches.—13 cm.

Internode length.—3.5-4.5 cm.

Diameter of branches.—7-8 mm.

Stem color.—Brownish-red, RHS 182 A.

Propagation.—Terminal tips for cuttings.

Rooting.—Roots initiate in about 18 days at 22° C., from sticking to transplanting.

Cultivation time.—It takes about 9-10 weeks of growing time to produce a marketable flowering plant in a 12 cm container.

Foliage:

Leaf arrangement.—Primarily in whorls.

Leaf shape.—Lanceolate to elliptic, unusually narrow, with acuminate base and acute to acuminate tip.

Surface.—Weakly glossy and almost smooth to slightly rippled.

Margin.—Weakly serrated, ciliated.

Leaf length.—13.0-14.5 cm.

Leaf width.—2.5-3.0 cm.

Upper surface color.—Deep medium green, uniform, no variegation, mature leaves RHS 137 B to 137 C, young leaves between RHS 137 C and 141 C.

Veins on upper surface.—Pink, RHS 54 B.

Lower surface color.—Light green, RHS 138 B (both young and mature leaves).

Veins on lower surface.—Purple, RHS 53 A.

Petiole.—20-30 mm in length, 3 mm in diameter.

Petiole color.—Upper side pink, RHS 54 A, lower side RHS 53 B.

### INFLORESCENCE

Flowering response.—9 weeks after planting of rooted cuttings.

Flowering season.—Generally indeterminate, mainly from March to October, depending on light intensity.

Flower:

Number of flowers per node.—8-10, in various stages of development.

Form of corolla.—Single-type, 5 petals.

Corolla shape.—Round, with the petals well overlapping, almost flat or very weakly cup-shaped.

Corolla size.—Average length: 72 mm Average width: 73 mm Depth of corolla: 10 mm.

Shape of petals.—Cordate, slightly lobed at the top end Top petal: 35 mm long, 52 mm wide Lateral petals 35 mm long, 33 mm wide Lower petals: 34 mm long, 45 mm wide Texture: Smooth, velvety Aspect: Mostly flat, occasionally somewhat wavy, banner petal may appear faintly cupped.

Color (general tonality from a distance of three meters).—Orange-red, brilliant, uniform.

Ground color of upper surface.—RHS 44 B, no markings.

Color of eye zone.—RHS 44 B.

Color of lower surface.—RHS 43 B.

Spur color.—Pink, RHS 53 C.

Spur shape and size.—Downwardly curved, 55-60 mm long, 3 mm in diameter at the flower end.

Pedicel length.—45-52 mm.

Pedicel color.—Reddish, RHS 53 B.

Flower bud.—Ovoid shape, 24 mm in length, 16 mm in width, main color RHS 42 A.

Reproductive organs:

Stamens.—5 in number, upper surface color is mainly RHS 44 A.

Anthers.—Fused, hooded.

Pollen.—Whitish-yellow, about RHS 8 D.

Style and stigma.—5 in number, very short, pale yellowish, about RHS 150 D.

Ovary.—5-celled, 5 mm long, surface color green, RHS 137 A.

Disease/Pest resistance/susceptibility: No observations to date.

I claim:

1. A new and distinct New Guinea Impatiens plant named ‘Fisnics Zorred’ as described and illustrated herein.
